(19) United States
(12) Patent Application Publication
(10) Pub. No.: US 2007/0283969 A1
(43) Pub. Date: Dec. 13, 2007

(54) Method of Diagnosing and Treating Erectile Dysfunction

(75) Inventors: Dwayne Yamasaki, Rohnert Park, CA (US); Mark Dolan, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/761,906

(22) Filed: Jun. 12, 2007

Related U.S. Application Data
(60) Provisional application No. 60/804,542, filed on Jun. 12, 2006.

(51) Int. Cl. A61F 2/26; A61F 2/92

(52) U.S. Cl. 128/898; 623/23.7

(57) ABSTRACT

A method of diagnosing and treating a patient having erectile dysfunction by placement of a stent or paving material within a stenosed region of a pelvic vessel. The diagnosing of the patient's condition includes determining if a treatable stenosis exists within a pelvic vessel and if proper blood flow is otherwise occurring within the penile arteries.

### CROSS-REFERENCE TO RELATED APPLICATIONS

This application claims the benefit under 35 U.S.C. § 119(e) of provisional application U.S. Appl. No. 60/804,542, filed Jun. 12, 2006, the disclosure of which is incorporated by reference herein in its entirety.

### FIELD OF THE INVENTION

The present invention relates to a method of diagnosing and treating erectile dysfunction. More particularly, a method of diagnosing a patient having erectile dysfunction caused by atherosclerosis into or through the common and internal iliac arteries and internal pudendal arteries with otherwise proper or moderately compromised flow within the cavernosal vessels for treatment by placement of a vessel wall support structure within the stenosed region of the affected vessels.

### BACKGROUND OF THE INVENTION

The National Institutes of Health estimates that 30 million American men suffer from mild, moderate or complete erectile dysfunction. Erectile dysfunction is the chronic, i.e., greater than three months duration, inability to maintain a penile erection sufficient to achieve penetration of one's sexual partner, complete sexual intercourse and/or achieve sexual satisfaction.

There are psychological and physical causes of erectile dysfunction. Most causes of erectile dysfunction have an adverse effect on nerves and/or blood vessels to, from, and within the penis. Vascular disease is a leading physical cause of erectile dysfunction. Atherosclerosis of the penile arteries alone may account for about 40% of patients over 40 years of age with erectile dysfunction. Other possible vascular-related causes include diabetes, hypertension, high cholesterol, renal disease, and smoking. It has been shown that causes of erectile dysfunction include stenoses anywhere along the arterial tree from the abdominal aorta to the helicine arteries. These arteries include, from proximal to distal, the distal abdominal aorta, common iliac, internal iliac (hypogastric), internal pudendal, and penile arteries, i.e., dorsal, deep, and helicine arteries.

Other causes of erectile dysfunction include the external iliac “steal syndrome”. This occurs when occlusion of the common iliac or inferior abdominal aorta leads to retrograde flow, i.e., flow in the opposite direction, in the internal iliac artery due to the rheological demand from the external iliac/femoral systems in the lower limb. Erectile dysfunction can also occur from stenosis resulting from radiation treatment for prostate cancer.

VIAGRA® (sildenafil citrate, Pfizer, Inc., New York, N.Y.), and other PDE-5 inhibitors, taken orally, is effective in a majority of patients to produce an adequate erection for sexual intercourse, but its effectiveness is dependent upon the severity of erectile dysfunction and/or any underlying disease. PDE-5 inhibitors have no proven effect on penile atherosclerosis, a primary cause of erectile dysfunction. PDE-5 inhibitors may be ineffective when, for instance, the degree of atherosclerosis is too large such that, after sexual stimulation, the arteries are not sufficiently compliant resulting in the amount of arterial dilation being inadequate with a corresponding insufficient amount of blood then flowing into the erectile chambers to produce an erection satisfactory for sexual intercourse.

In cases where PDE-5 inhibitors are not effective, a second drug, ALPROSTADIL® (Caverjet, Edex, Schwarz Pharma USA Holdings, Inc., Wilmington, Del.) has been shown to be effective. ALPROSTADIL®'s main disadvantage, however, is that it must be injected into the base of the penis into the corpora cavernosa with a needle or inserted into the urethra in pellet form through a delivery system called MUSE (Medicated Urethral Suppository for Erection). Also, an inappropriate dose of either of the afore-mentioned drugs can lead to priapism, or a prolonged erection not due to sexual arousal. Priapism beyond 6 to 8 hours can cause permanent damage to the penis, and requires immediate treatment.

A non-invasive treatment for erectile dysfunction is a hand-held vacuum pump, such as the Vacuum Erection Device by Osbon Medical Systems. A plastic cylinder (connected to the pump) is placed over the penis and a partial vacuum is quickly achieved, after pumping several times, which draws blood into the penis thereby dilating the vessels to achieve an artificial erection. A rubber constricting ring is placed temporarily at the base of the penis to prevent blood from escaping from the penis. The constricting ring may remain in place for not more than thirty minutes or damage to the penis may result.

When other therapies are ineffective, surgical implants may be considered. One device consists of two silicone rods implanted in the penis. The penis is permanently erect and can be pointed down along the thigh or up toward the abdomen to conceal it under clothing. Another type of implant consists of two inflatable cylinders put into the corpora cavernosa which produce an erection when filled with saline fluid. The fluid is pumped from a reservoir implanted in the space of Retsius and then fluid is transferred to the penile cylinders when an erection is desired. The aforementioned surgical implants have their obvious disadvantages.

Erectile dysfunction also occurs in men, with otherwise properly functioning penile arteries, due to atherosclerosis in a pelvic artery that is not adequately addressed by the aforementioned methods of treatment. Balloon angioplasty of an obstruction of an internal iliac and/or internal pudendal artery has been suggested to treat vasculogenic impotence but angioplasty has not been developed into a treatment option for erectile dysfunction. Thus additional methods of diagnosing and treating erectile dysfunction that is caused by insufficient flow to the penile arteries due to atherosclerosis of a pelvic artery are still needed.

### BRIEF SUMMARY OF THE INVENTION

An embodiment of the present invention is a method of diagnosing erectile dysfunction correctable by arterial stenting or paving. The method includes identifying a stenosis in a pelvic artery that is restricting blood flow to the penile arteries and imaging blood flow in the penis to ascertain flow velocity. The stenosis in the pelvic artery may be identified by performing a pelvic angiogram on the patient. The blood flow, viz., blood velocity, in the penis may be imaged by injecting ALPROSTADIL® into the base of the corpus cavernosum and using a color Doppler imaging device to measure the subsequent blood velocity. The method may further include analyzing the flow velocity of the penile cavernosum to determine whether blood flow within the penile arteries is sufficient to produce an erection.

In another embodiment, a diagnosis according to the present invention will help to identify patients having erectile dysfunction that will benefit from revascularizing the pelvic vessels by excluding patients that have pelvic neuropathies, severe arteriovenous fistulas in the cavernosum, and/or severe atherosclerosis in the cavernosum.

Another embodiment of the present invention is a method of treating erectile dysfunction caused by atherosclerosis in a pelvic artery. The method includes tracking a delivery catheter to a stenosis within the pelvic artery and delivering a vessel wall support structure, such as a stent or paving material, within the stenosis to restore flow through the pelvic artery to the penile arteries. In an embodiment, the pelvic artery is the internal pudendal artery that is reached by tracking the stent delivery catheter through the femoral artery, the internal iliac artery and then into the pudendal artery to the site of the stenosis. Another approach is to enter through the brachiocephalic artery (in the arm) and tracking down the descending aorta to the internal iliac.

In various other embodiments of diagnosis and treatment of a pelvic artery in accordance with the present invention, the pelvic artery may be one of the common iliac artery, external iliac artery, and/or internal iliac or hypogastric artery. In addition to stenting and paving, various treatments for revascularization of a pelvic artery in accordance with embodiments of the present invention include breaking-up and/or removing the stenosis by rotoblading, brachytherapy, ultrasound, clot retrieval, and/or drug/biologic delivery.

### BRIEF DESCRIPTION OF DRAWING

The foregoing and other features and advantages of the invention will be apparent from the following description of the invention as illustrated in the accompanying drawing. The accompanying drawing, which is incorporated herein and forms a part of the specification, further serves to explain the principles of the invention and to enable a person skilled in the pertinent art to make and use the invention. The drawing is not to scale.

FIG. 1 is an illustration of a guide catheter and stent delivery system for use in a method of treating erectile dysfunction according to an embodiment of the present invention.

### DETAILED DESCRIPTION OF EMBODIMENTS OF THE INVENTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

Erectile dysfunction is a disease that has several etiologies, one of which is insufficient blood flow to the penile arteries. Essentially, penile erection occurs when the two corpora cavernosa fill with blood and maintain pressure adequate for penetration. Each corpus cavernosum is fed by a deep artery of the penis located in the center of each cavernosum. The deep artery is a branch of the internal pudendal artery that is a branch of, and in some cases, a direct continuation of the internal iliac artery, also called the hypogastric arteries, which is a terminal branch of the common iliac arteries. The internal pudendal artery feeds most of the external genitalia and perineal structures, wherein the perineum refers to the diamond-shaped area between the thighs from the pubic bone to the tip of the coccyx.

Each deep artery has many smaller coil-shaped arteries, called helicine arteries extending downstream therefrom that open directly into the corpora cavernosa. Erection of the penis is a parasympathetic nervous system process that ultimately results in the release of the neurotransmitter, nitric oxide, and vasodilation of the helicine arteries in the corpora cavernosa. In addition to dilatation, the helicine arteries also straighten out from their normally coiled configuration to further increase blood flow into the cavernosa. Blood then fills these erectile compartments, and in the process compresses the penile veins that drain these tissues. Activation of the sympathetic nervous system returns the penis to a flaccid state.

Atherosclerosis occurring anywhere along the arterial path from the abdominal aorta through the internal pudendal artery can adversely affect blood flow to the penile arteries. As such, stenting within the pelvic arterial region may be effective in treating erectile dysfunction caused by the vascular disease, if the patient otherwise exhibits normal blood flow within the penis.

Whether the increased blood flow achievable by the revascularization procedure, such as stenting, results in effective treatment of the patient's erectile dysfunction depends on whether normal blood flow is otherwise occurring within the penis. In order to produce an erection, the blood flowing into the corpus cavernosa must reach a certain velocity and volume to achieve a pressure point great enough to cut-off the dorsal veins within the deep (Buck's) fascia of the penis and thereby minimize the flow of blood out of the penis. If the blood flow within the penis is insufficient to reach the pressure point, blood will continue to flow out of the penis making an erection unattainable. Among anatomical anomalies that may prevent the pressure from building sufficiently within the penis are the presence of fistula, which bypass the capillary connection between the deep arteries and dorsal veins within the penis that otherwise would slow blood flow to allow the requisite build-up of pressure, fibrosis of the corpus cavernosum, or neuropathy within the penis.

In accordance with an embodiment of the present invention, the initial step in making the diagnosis of whether a stenting or other revascularization procedure is likely to alleviate erectile dysfunction in the patient is to determine the blood flow characteristics within and proximate the deep artery of the cavernosa. In an embodiment, non-invasive Doppler imaging of the penis may be performed to determine the blood flow within the penis. Doppler imaging, e.g., ultrasound color Doppler imaging or echo imaging, may be used in the vasculature to determine flow velocity and direction, in other words, to determine how fast the blood is moving. Doppler imaging of the penis will aid in identifying whether blood flow through the cavernosum is sufficient to produce an erection. In another embodiment, a penile injection of ALPROSTADIL® or PAPAVERINE® or administration of another pharmacological stimulus alone might be sufficient to determine whether an erection can be created by increasing blood flow from the pelvic arteries. In other embodiments, penile functional capabilities may be accessed by tumescence monitoring, neurological stimulus of the pudendal nerve and/or neuropathic evaluation using temperature discrimination or biothesiometry.

In accordance with an embodiment of the present invention, a patient with erectile dysfunction that has been identified as having adequate blood flow characteristics within and proximate the deep artery of the cavernosa to produce an erection must than undergoe a diagnostic procedure in order to determine whether a revascularization procedure is likely to alleviate erectile dysfunction in the patient. As such, in this step of the diagnosis a pelvic angiogram may be performed on the patient. An angiogram is a diagnostic procedure using an x-ray to visualize blood vessels by following the introduction of a contrast material through an artery or system of vessels. A pelvic angiogram showing a stenosis in one of the pelvic arteries, such as the internal pudendal artery, may be treatable with a stent, a paving material or other revascularization procedure to increase blood flow to the penile arteries. In other embodiments of the present invention, other diagnostic procedures, such as a CT scan, MRI, CT-angiography (CTA), MR-angiography (MRA), indirectly via doppler or color duplex sonography, and nuclear imaging, i.e., radionuclide angiography, may be used to determine whether a stenosis exists in one of the pelvic arteries. In addition to stenting and paving, treatments for revascularization of a pelvic artery in accordance with embodiments of the present invention may include breaking-up and/or removing the stenosis by rotoblading, brachytherapy, ultrasound, clot retrieval, and/or drug/biologic delivery.

Upon diagnosis of a stentable or otherwise treatable stenosis within one of the patient's pelvic arteries, such as the internal pudendal artery, in conjunction with an otherwise proper or adequate flow of blood within the cavernosum arteries, treatment of the patient's erectile dysfunction may be effectuated by placement of or supporting structure. In accordance with various embodiments of the present invention, the vessel wall support device may be one of a self-expanding stent, a balloon expandable stent and a paving material that is placed within the stenosed region of the pelvic vasculature.

Due to the position of the internal pudendal artery within a man's anatomy, it may receive in vivo loading from forces external to the body that may potentially crush the internal pudendal artery, as well as any implanted stent therein, between the external force and the sacrospinous ligament. As such, if the stenosis to be treated is within the internal pudendal artery a self-expanding stent design may be warranted so that if subjected to acute high forces within the internal pudendal artery the self-expanding stent design will deform and then return to its original configuration. Thus in an embodiment of the present invention, a self-expanding stent may be implanted within the internal pudendal artery to provide vessel support within a stenosed region of the artery. The stent may be delivered to the treatment site by tracking a stent delivery catheter through an access site in the femoral artery then into the internal iliac artery and subsequently into the internal pudendal artery.

In addition, though contrary to conventional wisdom, recent testing performed by the inventors on a series of cadavers has proven that a balloon expandable stent design with sinusoidal elements was able to withstand direct external loading at the sacrospinous ligament and perineal loading directed at a balloon expanded stent implanted within the internal pudendal artery. Current balloon expanded stent designs would be desirable for use within the internal pudendal artery as, unlike self-expanding stents, they are readily available with outer expanded diameters that are small enough yet strong enough to be supportively implanted within the extremely narrow distal regions of the internal pudendal artery. Thus in another embodiment of the present invention, a balloon expandable stent may be implanted within the internal pudendal artery to provide vessel support within a stenosed region of the artery. The stent may be delivered to the treatment site by tracking a stent delivery catheter through an access site in the femoral artery then into the internal iliac artery and subsequently into the internal pudendal artery.

In an alternate embodiment, a catheter for use in a paving operation may be tracked to the treatment site and used to deliver a paving material to the stenosed region of the internal pudendal artery. Suitable paving materials that prevent restenosis and are anti-inflammatory include polymerizable hydrogels and photo polymerizable hydrogels, which may be laid down within the vasculature by methods and apparatus as described in U.S. Pat. No. 5,213,580 and U.S. Pat. No. 5,674,287, the disclosures of which are hereby incorporated by reference in their entireties.

In various other embodiments of diagnosis and treatment of a pelvic artery in accordance with the present invention, the pelvic artery may be one of the common iliac artery, and/or the internal iliac or hypogastric artery.

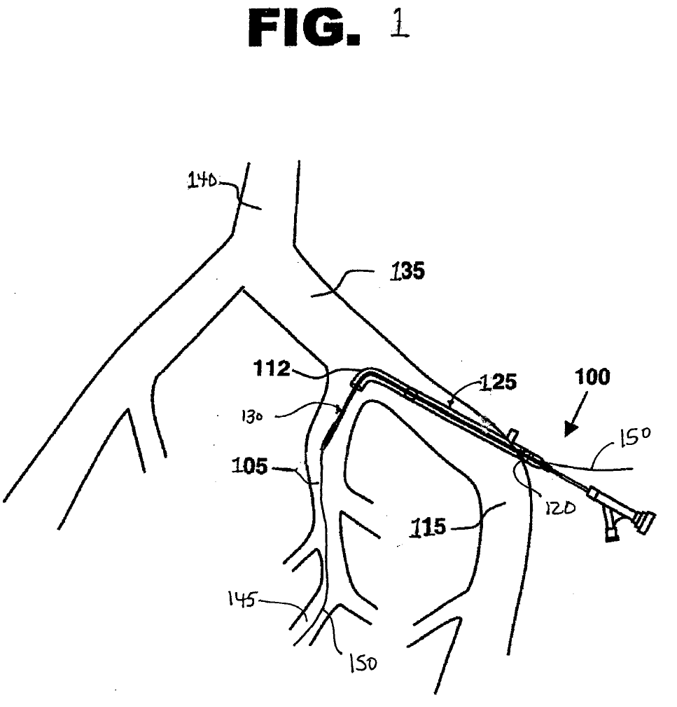

As shown in FIG. 1, a guide catheter and stent delivery system 100 may be used in embodiments of the present invention to more easily navigate the pelvic vasculature. System 100 is more fully disclosed in U.S. Patent Appl. Pub. No. US 2006/0079951 A1 to Dolan et al., which is incorporated by reference herein in its entirety. System 100 is introduced into femoral artery 115 through a percutaneous access site 120 to be tracked over a guidewire 150, which in FIG. 1, extends within the pelvic vasculature to the treatment site (not shown) within the internal pudendal artery 145. System 100 includes a guide catheter portion 125 shown passing within femoral artery 115 and having an angled tip 112 directed into an ostium of internal iliac artery 105. A stent delivery catheter portion 130 of system 100 is shown extending into internal iliac artery 105 from guide catheter portion 125. Stent delivery catheter portion 130 is then maneuverable through internal iliac artery 105 into internal pudendal artery 145 to the site of the stenosis (not shown). For reference, the right common iliac artery and abdominal aorta are indicated at 135 and 140, respectively. For an application as shown in FIG. 1, the length of guide catheter portion 125 of system 100 may be less than 50 centimeters. In order to access distal treatment sites of the internal pudendal artery, stent delivery catheter portion 130 may be positionable within a vessel diameter of as little as 2 mm in accordance with embodiments of the present invention. In alternative embodiments, a delivery catheter for delivering a paving material may be used instead of stent delivery catheter portion 130 and such a delivery catheter would also be operable within a vessel of as little as 2 mm.

A stent for use in embodiments of the present invention mentioned above may be balloon expandable or self-expanding and may be made from any suitable medically implantable material, such as, but not limited to, stainless steel, nitinol, tantalum, ceramic, nickel, titanium, aluminum, polymeric materials, cobalt alloys, magnesium alloys, platinum iridium, titanium ASTM F63-83 Grade 1, niobium, high carat gold K 19-22, and combinations thereof. Stents and structures for stents suitable for use in embodiments of the present invention are disclosed in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,935,162 to Dang, U.S. Pat. No. 6,090,127 to Globerman, and U.S. Pat. No. 6,730,116 to Wolinsky et al., each of which is incorporated by reference herein in its entirety. As well, any suitable guide catheter and/or stent delivery catheter may be utilized in embodiments of the present invention, with or without a guidewire, as would be apparent to one of ordinary skill in the art.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A method of diagnosing and treating erectile dysfunction, the method comprising the steps of:
verifying penile functional capability to have an erection;
identifying a stenosis in a pelvic artery that is restricting blood flow to the penile arteries; and
opening the identified stenosis in the pelvic artery with a stent.

2. The method of claim 1, wherein the step of identifying a stenosis in a pelvic artery includes performing a pelvic angiogram.

3. The method of claim 1, wherein the step of verifying penile functional capability is selected from the group consisting of using an ultrasound color Doppler imaging device and administering a penile injection.

4. The method of claim 1, wherein the stent used in the step of opening the identified stenosis is balloon expandable.

5. The method of claim 1, wherein the stent used in the step of opening the identified stenosis is self-expanding.

6. The method of claim 1, further comprising:
analyzing the flow velocity of the cavernosum for determining whether blood flow within the penis can produce an erection.

7. A method of treating erectile dysfunction caused by atherosclerosis in a pelvic artery, the method comprising:
tracking a delivery catheter to a stenosis within the pelvic artery; and
delivering a wall support device carried by the delivery catheter to the stenosis to restore flow through the pelvic artery.

8. The method of claim 7, wherein the pelvic artery is one of the internal pudendal artery, internal iliac artery and common iliac artery.

9. The method of claim 8, wherein the wall support device is a balloon expandable stent.

10. The method of claim 8, wherein the wall support device is a self-expanding stent.

11. The method of claim 8, wherein the wall support device is a vessel paving material.

12. A method of treating erectile dysfunction caused by atherosclerosis in a pelvic artery, the method comprising:
tracking a stent delivery catheter to a stenosis within the pelvic artery; and
delivering a stent to the stenosis to restore flow through the pelvic artery.

13. The method of claim 12, wherein the pelvic artery is one of the internal pudendal artery, internal iliac artery, and common iliac artery.

14. The method of claim 13, wherein the stent is a balloon expandable stent.

15. The method of claim 13, wherein the stent is a self-expanding stent.
